(19) United States
(12) Patent Application Publication
(10) Pub. No.: US 2023/0381208 A1
(43) Pub. Date: Nov. 30, 2023

(54) ORAL CANNABINOID FORMULATION COMPRISING MEDIUM CHAIN TRIGLYCERIDES AND TOCOPHERYL PHOSPHATES

(71) Applicant: AVECHO BIOTECHNOLOGY LIMITED, Clayton (AU)

(72) Inventor: Paul GAVIN, Clayton (AU)

(21) Appl. No.: 18/249,647

(22) PCT Filed: Oct. 18, 2021
(86) PCT No.: PCT/AU2021/051212

§ 371 Date: Apr. 19, 2023

(30) Foreign Application Priority Data
Oct. 19, 2020 (AU) 2020903781

(51) Int. Cl. A61K 31/00; A61K 47/14; A61K 47/22; A61K 9/00

(52) U.S. Cl. CPC A61K 31/658; A61K 9/0053; A61K 47/22; A61K 47/14

(57) ABSTRACT

The invention relates to an oral cannabinoid formulation comprising: a cannabinoid component comprising a cannabinoid; a carrier in the form of a medium chain triglyceride (MCT); and a tocopheryl phosphate component comprising mono-(tocopheryl) phosphate (TP) and di-(tocopheryl) phosphate (T2P) wherein the mass ratio of the cannabinoid to the tocopheryl phosphate component is about 10:1 to 1:10.

### FIELD OF THE INVENTION

The invention relates to oral cannabinoid formulations.

### BACKGROUND OF THE INVENTION

Cannabinoids have been proposed for use for a range of conditions including pain, inflammation, anxiety, depression, insomnia, sleep disorders, lack of energy, lack of alertness, weight gain, obesity, diabetes, metabolic syndrome, nausea (acute or anticipatory), epilepsy, spasticity, schizophrenia, bi-polar disorder, cancer and neoplasia, chronic pain, osteoarthritic pain, bacterial and/or fungal infection, fibromyalgia, appetite enhancement and/or appetite suppression.

Oral administration is a preferred route for administration of cannabinoids for some of these conditions.

One problem with oral cannabinoid formulations is that they tend to have sub-optimal absorption, characterised by sub-optimal bioavailability, cMax, tMax, duration of absorption and/or area under the plasma drug concentration-time curve (AUC) for a given dose.

Sub-optimal bioavailability may arise from the poor gastric solubility and absorption of the cannabinoids and/or the 1^{st} or 2^{nd} pass metabolism of oral cannabinoid formulations subsequent to ingestion.

US2019015329A discusses an oil-in-water emulsion formulation in which a primary surfactant comprising from 1 to 30% w/w of the formulation is utilised to form a stable dispersion of a hydrophobic phase comprising an active agent in water with a tocol phosphate.

US2006281716A discusses an alkaloid formulation comprising the reaction product of one or more alkaloids with one or more phosphate derivatives of one or more electron transfer agents.

US2015110924A discusses compositions that contain a modified food starch and one or more non-polar compounds. In some instances, the compositions contain a water-soluble derivative of vitamin E mixture, containing relatively high concentrations of dimer forms of the PEG-derivative of vitamin E.

US2021260143A discusses an oil in water microemulsion comprising a water solubility agent composition designed for preparing a MCT based oil phase in water microemulsion. The water solubility agent composition is based on using sucrose ester as emulsifier and lecithin as co-emulsifier.

WO21046628A1 discusses a cannabidiol composition for use in oral delivery comprising synthetic cannabidiol (CBD) having a purity of at least 99.8 percent, together with beta-caryophyllene (BCP) which functions as both a solvent and antioxidant. The compositions further contain at least one additional lipophilic solvent (e.g. medium chain triglycerides (MCT) and coconut oil) and at least one additional antioxidant (e.g. alpha tocopherol (vitamin E)).

US2021069103A discusses self-emulsifying drug delivery systems for oral delivery of cannabinoids. The cannabinoids are dissolved in an oily medium (e.g. medium chain triglycerides) together with at least one surfactant to improve dissolution, stability, and bioavailability.

US2021046438A discusses a CBD Nano-Emulsion material and process comprises a formulation comprising at least a lecithin or mixed lecithin, one or more carrier oils, and a Vitamin E TPGS from a sunflower version and a soy version.

US2020129463A discusses cannabidiol, beta-hydroxybutyrate, related compounds, including amino acids, short chain fatty acids, short chain triglycerides, medium chain fatty acids, medium chain triglycerides, long chain fatty acids, long chain triglycerides, berberine, metabolites of berberine (e.g., dihydroberberine), and/or combinations thereof to improve the health.

US2020138772A discusses formulations comprising a stabilized, aqueous purified cannabis oil emulsion comprising: a) CBD and THC wherein the ratio of CBD:THC by wt/wt is from 1,050:1 to 1:1,050, and b) at least one emulsifier selected from the group consisting of Poloxamer 188, Polysorbate 80, Polysorbate 20. Vit E-TPGS (TPGS), TPGS-1000, TPGS-750-M, Solutol HS 15, PEG-40 hydrogenated castor oil, PEG-35 Castor oil, PEG-8-glyceryl capylate/caprate, PEG-32-glyceryl laurate, PEG-32-glyceryl palmitostearate, Polysorbate 85, polyglyceryl-6-dioleate, sorbitan monooleate, Capmul MCM, Maisine 35-1, glyceryl monooleate, glyceryl monolinoleate, PEG-6-glyceryl oleate, PEG-6-glyceryl linoleate, oleic acid, linoleic acid, propylene glycol monocaprylate, propylene glycol monolaurate, polyglyceryl-3 dioleate, polyglyceryl-3 diisostearate and lecithin with and without bile salts, and mixtures thereof; and the uses in the treatment of diseases.

CN110638756A discusses a preparation is prepared from the following raw materials in parts by weight: 0.1-10 parts of cannabidiol, 5-15 parts of medium chain triglyceride (MOT), 1-15 parts of soybean lecithin, 1-20 parts of gamma-cyclodextrin, parts of glycerol-10 stearate, 10-45 parts of glycerol, and balance of distilled water. According to the preparation method, cannabidiol is prepared into the highly stable preparation through nano-encapsulation technology.

US2019104750A discusses coconut oil, coconut oil blends that are high in MCTs such as LouAna® liquid coconut oil, pure MOT oils, and Omega-3 oils may be emulsified to create an emulsified oil or blend. These oils and/or blends may be emulsified using an emulsifier that may be selected from the following: sunflower lecithin, sodium stearoyl lactylate (SSL), or a combination of sunflower lecithin and SSL.

US2007104741A discusses a self-emulsifying drug delivery system to improve dissolution, stability, and bioavailability of drug compounds of dronabinol or other cannabinoids. The drug compound(s) are dissolved in an oily medium (e.g. triglycerides and/or mixed glycerides and/or free fatty acids containing medium and/or long chain saturated, mono-unsaturated, and/or poly-unsaturated free fatty acids) together with at least one surfactant. The surfactant promotes self-emulsification, thereby promoting targeted chylomicron delivery and optimal bioavailability to a mammalian intestinal lumen.

US2002107265A discusses a pharmaceutical oil-in-water emulsions for delivery of polyfunctional active ingredients. The emulsions include an aqueous phase, an emulsifier, and an oil phase, wherein the oil phase includes a structured triglyceride that is substantially free of triglycerides having three C 6-C 12 fatty acid moieties, or a combination of a long chain triglyceride and a polarity-enhancing polarity modifier.

US2009005348A discusses a method of modulating one or more immuno-regulatory cytokines, such as pro-inflammatory and/or anti-inflammatory cytokines, comprising administering to a subject a therapeutically effective amount of one or more phosphate derivatives of one or more hydroxy chromans, or complexes thereof

US2009239827A discusses a therapy for lowering the blood levels of a lipid selected from the group comprising LDL cholesterol, triglycerides, overall cholesterol and mixtures thereof, the therapy comprising the step of administering an effective amount of one or more phosphate derivatives of one or more electron transfer agents.

US2009233881A discusses a method for alleviating symptoms, treating or preventing cancer, the method comprising administering to a subject, having or at risk of developing cancer, a pharmaceutical formulation comprising an effective amount of one or more phosphate derivatives of one or more hydroxy chromans selected from the group consisting of 7:8 dimethyl 6 hydroxy chromans, 8 methyl 6 hydroxy chromans and mixtures thereof.

US2009004166A discusses a carrier for use in enteral administration of biologically active compounds, said carrier comprising an effective amount of one or more phosphate derivatives of one or more electron transfer agents.

US2006241085A discusses a method of inhibiting the occurrence of one of more of the following conditions: —the proliferation of monocytes/macrophages; or —the proliferation of smooth muscle cells; or —the expression of 0036 receptors; or —the uptake of oxidized LDL, the method comprising the step of administering an effective amount of one or more phosphate derivatives of one or more electron transfer agents.

US2006257459A discusses a method for improving the efficacy and/or transdermal transport of topically administered pharmaceuticals and pharmacologically active compounds, said method comprising the step of incorporating the pharmaceutical or pharmacologically active compound in a carrier comprising an effective amount of one or more complexes of a phosphate derivative of a lipophilic pharmaceutically acceptable compound.

US2005009787A discusses a dietary or health supplement comprising an effective amount of a micronutrient selected from the group consisting of phosphate derivatives of tocopherol, ubiquinol, ascorbic acid, tocotrienol, retinal and mixtures thereof delivered with an acceptable carrier.

There is a need for oral cannabinoid formulations that provide for improvements in cannabinoid bioavailability.

### SUMMARY OF THE INVENTION

The invention relates to oral cannabinoid formulations, to use of same for providing improved bioavailability of cannabinoid to an individual requiring same, use of the formulations for treatment of a condition and to methods of manufacture of the oral cannabinoid formulations.

Various (enumerated) embodiments of the present invention are described herein. It will be understood that features specified in each embodiment may be combined with other specified features to provide further embodiments of the present disclosure.

Embodiment 1: An oral cannabinoid formulation, preferably a liquid formulation, more preferably a liquid oil formulation comprising:
a cannabinoid component comprising:
   a cannabinoid, preferably a synthetic cannabinoid, more preferably a synthetic cannabidiol (herein CBD) and/or synthetic tetrahydrocannabinol (herein THC), the cannabinoid in an amount to provide the oral cannabinoid formulation with a concentration of cannabinoid of about 1-250 mg, or 10-250 mg cannabinoid/ml of oral cannabinoid formulation;
   a carrier in the form of medium chain triglyceride (herein MCT), preferably a naturally occurring MCT extract or oil, more preferably a naturally occurring MCT extract or oil that comprises linear or branched alkyl chains comprising no more than about 12 carbon atoms;
   preferably wherein the mass ratio of cannabinoid to carrier is about 1:3 to about 1:1000 respectively, or about 1:3 to about 1:500 respectively, or about 1:3 to about 1:100 respectively; and
a tocopheryl phosphate component comprising:
   mono-(tocopheryl) phosphate (herein TP) and di-(tocopheryl) phosphate (herein T2P), preferably wherein the mass ratio of TP to T2P is about 6:4 to 8:2, preferably about 2:1 respectively, preferably wherein the TP and T2P are added to the formulation as acid forms of tocopheryl phosphates;
   optionally a solvent for increasing the solubility of the tocopheryl phosphates component;
      and wherein the mass ratio of the cannabinoid to the tocopheryl phosphate component is about 10:1 to 1:10, preferably 5:1 to 1:5, more preferably 2:1 to 1:2; and
optionally an aqueous component;
optionally wherein the formulation is provided in the form of a plurality of dosage units adapted for oral administration, each dosage unit comprising an amount of cannabinoid of about 1 to 250 mg,
   preferably wherein the oral cannabinoid formulation does not comprise dronabinol, or does not comprise a surfactant, or comprises less than 1% by weight alcohol.

Embodiment 2: A method for producing an oral cannabinoid formulation, preferably a liquid formulation, more preferably a liquid oil formulation, optionally further comprising an aqueous component, the method comprising the step of:
combining
   a tocopheryl phosphate component comprising:
      TP and T2P, preferably wherein the mass ratio of TP and T2P is about 6:4 to 8:2, preferably 2 to 1 respectively, preferably wherein the TP and T2P are added to the formulation as an acid form of tocopheryl phosphate;
      optionally a solvent for increasing the solubility of the tocopheryl phosphate component; with
   a cannabinoid component comprising:
      a cannabinoid, preferably a synthetic cannabinoid, more preferably a synthetic CBD and/or synthetic THC, the cannabinoid in an amount to provide the oral cannabinoid formulation with a concentration of cannabinoid of about 1-250 mg, or 10-250 mg cannabinoid/ml of oral cannabinoid formulation;
      a carrier in the form of MCT, preferably a naturally occurring MCT extract or oil, more preferably a naturally occurring MCT extract or oil that comprises linear or branched alkyl chains comprising no more than about 12 carbon atoms;
      preferably wherein the mass ratio of cannabinoid to carrier is about 1:3 to about 1:1000 respectively, or about 1:3 to about 1:500 respectively, or about 1:3 to about 1:100 respectively;
         to produce an oral cannabinoid formulation comprising a cannabinoid and a tocopheryl phosphate component in a mass ratio of about 10:1 to 1:10, preferably 5:1 to 1:5, more preferably 2:1 to 1:2 respectively;
         preferably wherein the oral cannabinoid formulation does not comprise dronabinol, or does not comprise a surfactant, or comprises less than 1% by weight alcohol.

Embodiment 3: A kit for producing an oral cannabinoid formulation of Embodiment 1, the kit comprising:
a tocopheryl phosphate component comprising:
   TP and T2P, preferably wherein the mass ratio of TP and T2P is about 6:4 to 8:2, preferably 2 to 1 respectively, preferably wherein the TP and T2P are added to the formulation as an acid form of tocopheryl phosphate;
   optionally a solvent for increasing the solubility of the tocopheryl phosphate component;
written instructions for utilising the tocopheryl phosphate component in the method of Embodiment 2;
optionally
a cannabinoid component comprising:
   a cannabinoid, preferably a synthetic cannabinoid, more preferably a synthetic CBD and/or synthetic THC, the cannabinoid in an amount to provide an oral cannabinoid formulation with a concentration of cannabinoid of about 1-250 mg, or about 10-250 mg cannabinoid/ml of oral cannabinoid formulation;
the optional cannabinoid component being separate from or combined with the tocopheryl phosphate component in the kit.

The kit of Embodiment 3 may further comprise, separate from the tocopheryl phosphate component, and the optional cannabinoid component, a carrier in the form of MCT, preferably a naturally occurring MCT extract or oil, more preferably a naturally occurring MCT extract or oil that comprises linear or branched alkyl chains comprising no more than about 12 carbon atoms.

Embodiment 4: An oral cannabinoid formulation, preferably a liquid formulation, more preferably a liquid oil formulation comprising:
a cannabinoid component comprising:
   a cannabinoid, preferably a synthetic cannabinoid, more preferably a synthetic CBD and/or synthetic THC, the cannabinoid in an amount to provide the oral cannabinoid formulation with a concentration of cannabinoid of about 1-250 mg, or 10-250 mg cannabinoid/ml of oral cannabinoid formulation;
   a carrier in the form of MCT, preferably a naturally occurring MCT extract or oil, more preferably a naturally occurring MCT extract or oil that comprises linear or branched alkyl chains comprising no more than about 12 carbon atoms;
   preferably wherein the mass ratio of cannabinoid to carrier is about 1:3 to about 1:1000 respectively, or about 1:3 to about 1:500 respectively, or about 1:3 to about 1:100 respectively; and
a tocopheryl phosphate component comprising:
   TP and T2P, preferably wherein the mass ratio of TP to T2P is about 6:4 to 8:2, preferably about 2:1 respectively, preferably wherein the TP and T2P are added to the formulation as acid forms of tocopheryl phosphates;
   optionally a solvent for increasing the solubility of the tocopheryl phosphates component;
      and wherein the mass ratio of the cannabinoid to the tocopheryl phosphate component is about 10:1 to 1:10, preferably 5:1 to 1:5, more preferably 2:1 to 1:2;
optionally wherein the formulation is provided in the form of a plurality of dosage units adapted for oral administration, each dosage unit comprising an amount of cannabinoid of about 1 to 250 mg, or 10 to 250 mg,
   preferably wherein the oral cannabinoid formulation comprises about 2.5% or less by weight water.

Embodiment 5: A capsule for oral consumption comprising:
a cannabinoid oil comprising:
   a cannabinoid, preferably a synthetic cannabinoid, more preferably a synthetic CBD and/or synthetic THC, the cannabinoid in an amount to provide the capsule with about 1-250 mg, or about 10-250 mg of cannabinoid;
      wherein the cannabinoid is provided in a carrier in the form of MCT, preferably a naturally occurring MCT extract or oil, more preferably a naturally occurring MCT extract or oil that comprises linear or branched alkyl chains comprising no more than about 12 carbon atoms;
   a tocopheryl phosphate component in the form of TP and T2P, preferably wherein the mass ratio of TP to T2P is about 6:4 to 8:2, preferably about 2:1 respectively, preferably wherein the TP and T2P are added to the formulation as acid forms of tocopheryl phosphates;
   wherein the mass ratio of the cannabinoid to the tocopheryl phosphate component is about 10:1 to 1:10, preferably 5:1 to 1:5, more preferably 2:1 to 1:2; and
   optionally a further component selected from the group consisting of an emulsifier, a buffering agent, an aqueous solvent, an anti-oxidant and a rheology modifier,
a hydrogel, preferably gelatin for encapsulating the cannabinoid oil;
preferably wherein the capsule comprises about 2.5% or less by weight water.

Embodiment 6: A method for providing an individual with a plasma concentration of cannabinoid, the method comprising the step of:
oral administration of a treatment formulation to an individual, the treatment formulation according to any one of Embodiments 1 or 4 to 5,
   wherein the plasma concentration of cannabinoid provided in the individual by oral administration of the treatment formulation is greater than that obtained by oral administration of a control formulation, wherein the control formulation is the same as the treatment formulation but does not comprise the tocopheryl phosphate component of the treatment formulation.

Embodiment 7: A method of increasing the half-life of a cannabinoid in plasma of an individual, the method comprising the step of:
oral administration of the formulation of Embodiments 1 or 4 to 5 to the individual,
   wherein the half-life of a cannabinoid in plasma of the individual by oral administration of the formulation of Embodiments 1 or 4 to 5 is greater than that obtained by administration of a same or similar dose of cannabinoid in a formulation that does not comprise the tocopheryl phosphate component.

Embodiment 8: A method of increasing the duration of a therapeutically effective plasma concentration of a cannabinoid in plasma of an individual, the method comprising the step of:
oral administration of a treatment formulation to an individual, the treatment formulation according to any one of Embodiments 1 or 4 to 5,
   wherein the duration of a therapeutically effective plasma concentration of a cannabinoid in plasma in the individual by oral administration of the treatment formulation is greater than that obtained by oral administration of a control formulation, wherein the control formulation is the same as the treatment formulation but does not comprise the tocopheryl phosphate component of the treatment formulation.

Embodiment 9: A method for treating an individual for a condition, preferably a condition selected from the group consisting of conditions including pain, inflammation, anxiety, depression, insomnia, sleep disorders, lack of energy, lack of alertness, weight gain, obesity, diabetes, metabolic syndrome, nausea (acute or anticipatory), epilepsy, spasticity, schizophrenia, bi-polar disorder, cancer and neoplasia, chronic pain, osteoarthritic pain, bacterial and/or fungal infection, fibromyalgia, appetite enhancement and/or appetite suppression, the method comprising step of:
oral administration of a therapeutically effective amount of a cannabinoid formulation of any one of Embodiments 1 or 4 to 5.

Embodiment 10: An oral cannabinoid formulation of Embodiments 1 or 4 to 5 for use in preventing or treating an individual for a condition, preferably a condition selected from the group consisting of conditions including pain, inflammation, anxiety, depression, insomnia, sleep disorders, lack of energy, lack of alertness, weight gain, obesity, diabetes, metabolic syndrome, nausea (acute or anticipatory), epilepsy, spasticity, schizophrenia, bi-polar disorder, cancer and neoplasia, chronic pain, osteoarthritic pain, bacterial and/or fungal infection, fibromyalgia, appetite enhancement and/or appetite suppression, preferably wherein the formulation comprises a therapeutically effective amount of a cannabinoid formulation of Embodiments 1 or 4 to 5

Embodiment 11: A method for providing an individual with an increased cMax or AUC of a cannabinoid comprising oral administration of a cannabinoid formulation of Embodiments 1 or 4 to 5 to an individual in need of said increased cMax or AUC, wherein the cMax or AUC of cannabinoid in an individual to whom the formulation of Embodiments 1 or 4 to 5 has been orally administered is increased relative to the cMax or AUC of cannabinoid arising from oral administration of a cannabinoid formulation that does not comprise the tocopheryl phosphate component.

### BRIEF DESCRIPTION OF THE FIGURES

FIG. 1. CBD solubility in-vitro in simulated gastric (0-30 min) and intestinal (30-90 min) conditions. Group 1: MCT. Group 2: 50 mg/ml TPM in MCT. Group 3: 100 mg/ml TPM in MCT.

FIG. 2—Mean CBD Cmax after a single oral gavage of formulations containing CBD.

FIG. 3—Mean CBD AUC after a single oral gavage of formulations containing CBD

### DETAILED DESCRIPTION OF THE INVENTION

### 1. Definitions

For the purpose of interpreting this specification, the following definitions will apply and whenever appropriate, terms use in the singular will also include the plural and vice versa.

As used herein, the term “about” in relation to a numerical value X means+/−10%, unless the context dictates otherwise.

As used herein, the term “pharmaceutically acceptable” means a non-toxic material that does not interfere with the effectiveness of the biological activity of the active ingredient(s).

As used herein, the term “treat”, “treating” or “treatment” in connection to a disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, {e.g., stabilization of a discernible symptom), physiologically, {e.g., stabilization of a physical parameter), or both. The term “alleviating” or “alleviation”, for example in reference to a symptom of a condition, as used herein, refers to reducing at least one of the frequency and amplitude of a symptom of a condition in a patient. In one embodiment, the terms “method for the treatment” or “method for treating”, as used herein, refer to “method to treat”.

As used herein, the term “therapeutically effective amount” refers to an amount of cannabinoid which is sufficient to achieve the stated effect. Accordingly, a therapeutically effective amount of cannabinoid will be an amount sufficient for the treatment or prevention of the relevant condition.

By “therapeutic regimen” is meant the pattern of treatment of an illness, e.g., the pattern of dosing used during the treatment of the disease or disorder.

As used herein, a subject or individual is “in need of” a treatment if such subject would benefit biologically, medically or in quality of life from such treatment. An individual is generally a mammal, typically a human, and may be a companion animal, livestock or performance animal.

The words “comprise”, “comprises”, “comprising” and “comprised” are used in an inclusive sense, unless the context requires otherwise.

### 2. Modes of Carrying Out the Invention

2.1 Formulations

The invention provides an oral cannabinoid formulation comprising:
a cannabinoid component comprising:
   a cannabinoid;
   a carrier in the form of medium chain triglyceride (herein MCT);
a tocopheryl phosphate component comprising:
   mono-(tocopheryl) phosphate (herein TP) and di-(tocopheryl) phosphate (herein T2P);
      wherein the mass ratio of the cannabinoid to the tocopheryl phosphate component is about 10:1 to 1:10, preferably 5:1 to 1:5, more preferably 2:1 to 1:2.

A cannabinoid may be a synthetic compound or a naturally occurring compound, for example a phyto-cannabinoid. Neutral cannabinoids include cannabigerol (CBG) and related compounds (e.g., cannabigerol monomethyl ether, cannabigerovarin); cannabichromene (CBC) and related compounds (e.g., (±)-cannabichromene, (±)-cannabichromevarin); (−)-cannabidiol (CBD) and related compounds (e.g., cannabidiol momomethyl ether, cannabidiol-C4, 20 (−)-cannabidivarin, cannabidiorcol); cannabinodiol (CBND) and related compounds (e.g., cannabinodivarin); Δ9-tetrahydrocannabinol (THC) and related compounds (e.g., Δ9-tetrahydrocannabinol-C4, Δ9-tetrahydrocannabivarin, Δ9-tetrahydro-cannabiorcol, (−)-Δ8-trans-(6aR,10aR)-Δ8-tetrahydrocannabinol, (−)-(6aS,10aR)-Δ9-tetrahydrocannabinol); cannabinol (CBN) and related compounds (e.g., cannabinol-C4, cannabivarin, cannabinol-C2, cannabiorcol, 25 cannabinol methyl ether); (±)-cannabitriol (CBT) and related compounds (e.g., (−)-(9R,10R)-trans-10-O-ethyl-cannabitriol, (±)-(9R,10R/9S,10S)-cannabitriol-C3); cannabielsoin (CBE) and related compounds (e.g., (5aS,6S,9R,9aR)-cannabielsoin, (5aS,6S,9R,9aR)-C3-cannabielsoin, cannabiglendol-C3, dehydrocannabifuran, cannabifuran); isocannabinoids (e.g., (−)-Δ7-trans-(1R,3R,6R)-isotetrahydrocannabinol, (±)-Δ7-1,2-cis-(1R,3R,6S)-isotetrahydrocannabivarin, (±)-Δ7-1,2-cis-(1 S,3S,6R)-isotetrahydrocannabivarin, (−)-Δ7 30-trans-(1R,3R,6R)-isotetrahydrocannabivarin); cannabicyclol (CBL) and related compounds (e.g., (±)-(1 aS,3aR,8bR,8cR)-cannabicyclol CBL-05, (±)-(1aS,3aR,8bR,8cR)-cannabicyclovarin); cannabicitran (CBT) and related compounds; and cannabichromanone (CBCN) and related compounds (e.g., cannabichromanone-C3, cannabicoumaronone). Acidic cannabinoids include cannabigerolic acid A; cannabigerolic acid A monomethyl ether; cannabigerovarinic acid A; (±)-cannabichromenic acid A; (±)-cannabichromevarinic acid A; cannabidiolic acid; cannabidivarinic acid; Δ9-tetrahydrocannabinolic acid A; Δ9-tetrahydrocannabinolic acid B; Δ9-tetrahydrocannabinolic acid C4 A; Δ9-tetrahydrocannabinolic acid-C4 B; Δ9-tetrahydro-cannabivarinic acid A; Δ9 5-tetrahydrocannabiorcolic acid A; Δ9-tetrahydrocannabiorcolic acid B; (−)-Δ8-trans-(6aR,10aR)-tetrahydrocannabinolic acid A; cannabinolic acid A; (5aS,6S,9R,9aR)-cannabielsoic acid A; (5aS,6S,9R,9aR)-cannabielsoic acid B; (5aS,6S,9R,9aR)-C3-cannabielsoic acid B; and (±)-(1aS,3aR,8bR,8cR)-cannabicyclolic acid A.

In one embodiment, the formulation comprises a heterogenous mixture of cannabinoid compounds. Preferably the formulation comprises at least cannabidiol (herein CBD) and/or tetrahydrocannabinol (herein THC).

The cannabinoid may be provided as an extract of a naturally occurring source of cannabinoid. More preferably the extract may comprise CBD and THC. Extracts of a naturally occurring source of cannabinoid may be obtained by extraction processes known to the skilled worker for extraction of phytocannabinoids, such as alcohol extraction, CO₂ extraction or other solvent free extraction. An extract may take the form of an oil.

A cannabinoid may be predominantly a single compound, for example CBD or THC, as obtained by fractionation of an extract of a natural source of cannabinoid, or by cannabinoid synthesis.

A cannabinoid may be a synthetic cannabinoid such as dronabinol. In this embodiment, the oral cannabinoid formulation may not comprise a surfactant, or may not comprise more than about 1% by mass of an alcohol.

In one embodiment the cannabinoid is provided as a racemic mixture (i.e. having both D & L stereochemistries), for example as obtainable by extraction of a natural source of cannabinoid.

In one embodiment, the cannabinoid component comprises cannabidiol (herein CBD) in an amount of about 1 to 250 mg/ml, preferably 10 to 100 mg/ml, preferably about 75 mg/ml of the formulation.

In one embodiment, the cannabinoid component comprises tetrahydrocannabinol (herein THC) in an amount of about 1 to 50 mg/ml, preferably 20 to 40 mg·ml.

A cannabinoid component of the formulation may comprise CBD and THC in a ratio of about 1:1, 2:3, 4:1 or 1:20. In another embodiment the ratio of CBD to THC may be 5:1 or 10:1.

In one embodiment, the cannabinoid component may further comprise other components commonly found in a naturally derived cannabinoid product such as a terpene.

The MCT may be obtained from a naturally occurring source, or it may be synthetic.

The MCT may be provided as a naturally occurring MCT extract or oil. Examples of oils include palm kernel oil and coconut oil.

Typically, the MCT comprises linear or branched alkyl chains comprising no more than about 12 carbon atoms.

The MCT may be a naturally occurring oil or extract that has been purified or fractionated thereby increasing the relative abundance of one or more linear or branched alkyl chains comprising 12 or less carbon atoms in the oil or extract. For example, the MCT may be derived from a plant oil such as palm kernel oil or coconut oil. The oil is fractionated or otherwise processed so that the amount of a given fatty acid, for example, 6:0 (caproic), 8:0 (caprylic), 10:0 (capric), 12:0 (lauric) acid chain has a higher relative amount in the fractionated or processed oil than is observed in the plant oil from which the fractionated or processed oil is derived. In a preferred embodiment, the fatty acids of the fractionated or processed oil may consist of the following fatty acid chains in the following stated amounts: caproic acid (6%), caprylic acid (55-85%), capric acid (15-40%), lauric acid (4%).

The MCT may consist of saturated fatty acid chains.

The MCT may comprise or consist of one or more fatty acid chains selected from the group consisting of caproic acid, caprylic acid, capric acid and lauric acid.

The MCT may consist of unsaturated fatty acid chains, for example fatty acid chains having one or more double bonds.

The MCT may consist of a mixture of saturated fatty acid chains and unsaturated fatty acid chains, said fatty acid chains having 12 or less carbon atoms.

The MCT oil may consist of a mixture of tri-, di- and mono-glycerides, or a mixture of tri- and mono-glycerides, or a mixture of tri- and di-glycerides or a mixture di- and mono-glycerides, or tri-glycerides, or di-glycerides, or mono-glycerides. In a preferred embodiment the MCT oil consists of tri-, di-, and mono-glycerides that comprise fatty acid chains that are 6:0, 8:0, 10:0, or, 12:0 carbon chains.

The MCT may be obtained from commercial sources, examples including Labrafac CC, Wabrafac WL1349, Captex 300, Captex 355 as described in the examples herein.

The mass ratio of cannabinoid to carrier is about 1:3 to 1:1000, or about 1:3 to 1:500, or about 1:3 to 1:100 respectively.

Tocopheryl phosphate is a phosphorylated tocopherol compound, where a covalent bond is formed between an oxygen atom (typically originating from a hydroxyl group) of the tocopherol compound and the phosphorous atom of a phosphate group (PO₄).

The phosphorylated tocopherol compound may be a phosphate mono-ester, phosphate diester, phosphate tri-ester, pyrophosphate mono-ester, pyrophosphate di-ester, or a salt or 5 derivative thereof, or a mixture thereof.

Salts of tocopheryl phosphate may include metal salts such as alkali or alkaline earth metal salts, for example sodium, magnesium, potassium and calcium salts. Other pharmaceutically or veterinary acceptable salts of the tocopheryl phosphate may be used, such as other alkali metal salts. Other pharmaceutically acceptable salts are well known in the art, and include the acceptable salts 10 described in detail in S. M. Berge, et al., J. Pharmaceutical Sciences, 66:1-19, 1977. Sodium and potassium salts are preferred.

The tocopheryl phosphate may be selected from, but not limited to, a mono-(tocopheryl) phosphate, a mono-(tocopheryl) phosphate monosodium salt, a mono-(tocopheryl) phosphate disodium salt, a di-(tocopheryl) phosphate, a di-(tocopheryl) phosphate monosodium salt, or a mixture thereof.

It is preferred that the TP and T2P are added to the formulation as an acid form of tocopheryl phosphate.

In particular embodiments, the composition comprises a mixture of TP and T2P in mass ratio of at about 2:1, within the range of about 4:1 to about 1:4, or within the range of about 6:4 to about 8:2. In some embodiments, the ratio is about 6:4 or about 8:2.

As described further herein, the tocopheryl phosphate component may further comprise an organic solvent, such as an alcohol for increasing the solubility of the tocopheryl phosphate component in the cannabinoid component of the formulation.

The oral cannabinoid formulation may take the form of a liquid, solid or semi-solid.

The formulation may further comprise an aqueous component, or the formulation may be mixed with an aqueous component prior to oral administration.

Where the formulation further comprises an aqueous component, it may present as an emulsion, a colloidal suspension or a bi-phasic solution.

The formulation may further comprise components including thickeners, gelling agents, buffers, emollients, sweeteners, disintegrators, flavours, colours, electrolytes, pH modifiers, appearance modifiers, sustained-release agents, and the like. Such additional components may be added to either of the cannabinoid or tocopheryl phosphate components, during any step during the formulation process.

The formulation may be provided in the form of a plurality of dosage units adapted for oral administration.

A dosage unit adapted for oral administration of a formulation may comprise an amount of cannabinoid of about 1 to 250 mg/ml, or 10 to 250 mg/ml.

A dosage unit may be presented as a tablet, caplet, capsule or liquid adapted for oral administration such as a syrup, suspension or spray.

In one embodiment, the dosage unit is a ‘gummie’. A gummie may otherwise be known as a ‘gummy candy’ or ‘jelly sweet’. A gummie may be a gelatin-based chewable confectionery. A gummie may be sugar free or otherwise unsweetened.

In a first oil formulation embodiment there is provided a cannabinoid oil formulation for oral administration comprising: CBD (about 10% w/w), MCT (about 70% w/w), TPM (about 7.5% w/w), tromethamine (about 1.40% w/w), water (about 2.5% w/w), lecithin (about 7.5% w/w), simethicone (about 0.02%) and butylated hydroxytoluene (about 0.2% w/w).

In a second oil formulation embodiment there is provided a cannabinoid oil formulation for oral administration comprising: CBD (about 1% w/w), MCT (about 85% w/w), TPM (about 2% w/w), tromethamine (about 1.40% w/w), water (about 2.5% w/w), lecithin (about 7.5% w/w), simethicone (about 0.02%) and butylated hydroxytoluene (about 0.2% w/w).

In a third oil formulation embodiment there is provided a cannabinoid oil formulation for oral administration comprising: CBG (about 10% w/w), MCT (about 70% w/w), TPM (about 7.5% w/w), tromethamine (about 1.40% w/w), water (about 2.5% w/w), lecithin (about 7.5% w/w), simethicone (about 0.02%) and butylated hydroxytoluene (about 0.2% w/w).

In a fourth oil formulation embodiment there is provided a cannabinoid oil formulation for oral administration comprising: CBG (about 1% w/w), MCT (about 85% w/w), TPM (about 2% w/w), tromethamine (about 1.40% w/w), water (about 2.5% w/w), lecithin (about 7.5% w/w), simethicone (about 0.02%) and butylated hydroxytoluene (about 0.2% w/w).

In a fifth oil formulation embodiment there is provided a cannabinoid oil formulation for oral administration comprising: CBC (about 10% w/w), MCT (about 70% w/w), TPM (about 7.5% w/w), tromethamine (about 1.40% w/w), water (about 2.5% w/w), lecithin (about 7.5% w/w), simethicone (about 0.02%) and butylated hydroxytoluene (about 0.2% w/w).

In a sixth oil formulation embodiment there is provided a cannabinoid oil formulation for oral administration comprising: CBC (about 1% w/w), MCT (about 85% w/w), TPM (about 2% w/w), tromethamine (about 1.40% w/w), water (about 2.5% w/w), lecithin (about 7.5% w/w), simethicone (about 0.02%) and butylated hydroxytoluene (about 0.2% w/w).

In a seventh oil formulation embodiment there is provided a cannabinoid oil formulation for oral administration comprising: CBN (about 10% w/w), MCT (about 70% w/w), TPM (about 7.5% w/w), tromethamine (about 1.40% w/w), water (about 2.5% w/w), lecithin (about 7.5% w/w), simethicone (about 0.02%) and butylated hydroxytoluene (about 0.2% w/w).

In an eighth oil formulation embodiment there is provided a cannabinoid oil formulation for oral administration comprising: CBN (about 1% w/w), MCT (about 85% w/w), TPM (about 2% w/w), tromethamine (about 1.40% w/w), water (about 2.5% w/w), lecithin (about 7.5% w/w), simethicone (about 0.02%) and butylated hydroxytoluene (about 0.2% w/w).

In a ninth oil formulation embodiment there is provided a cannabinoid oil formulation for oral administration comprising: CBND (about 10% w/w), MCT (about 70% w/w), TPM (about 7.5% w/w), tromethamine (about 1.40% w/w), water (about 2.5% w/w), lecithin (about 7.5% w/w), simethicone (about 0.02%) and butylated hydroxytoluene (about 0.2% w/w).

In a tenth oil formulation embodiment there is provided a cannabinoid oil formulation for oral administration comprising: CBND (about 1% w/w), MCT (about 85% w/w), TPM (about 2% w/w), tromethamine (about 1.40% w/w), water (about 2.5% w/w), lecithin (about 7.5% w/w), simethicone (about 0.02%) and butylated hydroxytoluene (about 0.2% w/w).

In an eleventh oil formulation embodiment there is provided a cannabinoid oil formulation for oral administration comprising: THC (about 10% w/w), MCT (about 70% w/w), TPM (about 7.5% w/w), tromethamine (about 1.40% w/w), water (about 2.5% w/w), lecithin (about 7.5% w/w), simethicone (about 0.02%) and butylated hydroxytoluene (about 0.2% w/w).

In a twelfth oil formulation embodiment there is provided a cannabinoid oil formulation for oral administration comprising: THC (about 1% w/w), MCT (about 85% w/w), TPM (about 2% w/w), tromethamine (about 1.40% w/w), water (about 2.5% w/w), lecithin (about 7.5% w/w), simethicone (about 0.02%) and butylated hydroxytoluene (about 0.2% w/w).

In a thirteenth oil formulation embodiment there is provided a cannabinoid oil formulation for oral administration comprising: THCV (about 10% w/w), MCT (about % w/w), TPM (about 7.5% w/w), tromethamine (about 1.40% w/w), water (about 2.5% w/w), lecithin (about 7.5% w/w), simethicone (about 0.02%) and butylated hydroxytoluene (about 0.2% w/w).

In a fourteenth oil formulation embodiment there is provided a cannabinoid oil formulation for oral administration comprising: THCV (about 1% w/w), MCT (about % w/w), TPM (about 2% w/w), tromethamine (about 1.40% w/w), water (about 2.5% w/w), lecithin (about 7.5% w/w), simethicone (about 0.02%) and butylated hydroxytoluene (about 0.2% w/w).

In a fifteenth oil formulation embodiment there is provided a cannabinoid oil formulation for oral administration comprising: CBGA (about 10% w/w), MCT (about % w/w), TPM (about 7.5% w/w), tromethamine (about 1.40% w/w), water (about 2.5% w/w), lecithin (about 7.5% w/w), simethicone (about 0.02%) and butylated hydroxytoluene (about 0.2% w/w).

In a sixteenth oil formulation embodiment there is provided a cannabinoid oil formulation for oral administration comprising: CBGA (about 1% w/w), MCT (about % w/w), TPM (about 2% w/w), tromethamine (about 1.40% w/w), water (about 2.5% w/w), lecithin (about 7.5% w/w), simethicone (about 0.02%) and butylated hydroxytoluene (about 0.2% w/w).

In a seventeenth oil formulation embodiment there is provided a cannabinoid oil formulation for oral administration comprising: CBT (about 10% w/w), MCT (about 70% w/w), TPM (about 7.5% w/w), tromethamine (about 1.40% w/w), water (about 2.5% w/w), lecithin (about 7.5% w/w), simethicone (about 0.02%) and butylated hydroxytoluene (about 0.2% w/w).

In an eighteenth oil formulation embodiment there is provided a cannabinoid oil formulation for oral administration comprising: CBT (about 1% w/w), MCT (about 85% w/w), TPM (about 2% w/w), tromethamine (about 1.40% w/w), water (about 2.5% w/w), lecithin (about 7.5% w/w), simethicone (about 0.02%) and butylated hydroxytoluene (about 0.2% w/w).

In a nineteenth oil formulation embodiment there is provided a cannabinoid oil formulation for oral administration comprising: CBE (about 10% w/w), MCT (about 70% w/w), TPM (about 7.5% w/w), tromethamine (about 1.40% w/w), water (about 2.5% w/w), lecithin (about 7.5% w/w), simethicone (about 0.02%) and butylated hydroxytoluene (about 0.2% w/w)

In a twentieth oil formulation embodiment there is provided a cannabinoid oil formulation for oral administration comprising: CBE (about 1% w/w), MCT (about 85% w/w), TPM (about 2% w/w), tromethamine (about 1.40% w/w), water (about 2.5% w/w), lecithin (about 7.5% w/w), simethicone (about 0.02%) and butylated hydroxytoluene (about 0.2% w/w).

In a twenty-first oil formulation embodiment there is provided a cannabinoid oil formulation for oral administration comprising: CBL (about 10% w/w), MCT (about 70% w/w), TPM (about 7.5% w/w), tromethamine (about 1.40% w/w), water (about 2.5% w/w), lecithin (about 7.5% w/w), simethicone (about 0.02%) and butylated hydroxytoluene (about 0.2% w/w).

In a twenty-second oil formulation embodiment there is provided a cannabinoid oil formulation for oral administration comprising: CBL (about 1% w/w), MCT (about 85% w/w), TPM (about 2% w/w), tromethamine (about 1.40% w/w), water (about 2.5% w/w), lecithin (about 7.5% w/w), simethicone (about 0.02%) and butylated hydroxytoluene (about 0.2% w/w).

In a twenty third oil formulation embodiment there is provided a cannabinoid oil formulation for oral administration comprising: CBCN (about 10% w/w), MCT (about % w/w), TPM (about 7.5% w/w), tromethamine (about 1.40% w/w), water (about 2.5% w/w), lecithin (about 7.5% w/w), simethicone (about 0.02%) and butylated hydroxytoluene (about 0.2% w/w).

In a twenty fourth oil formulation embodiment there is provided a cannabinoid oil formulation for oral administration comprising: CBCN (about 1% w/w), MCT (about % w/w), TPM (about 2% w/w), tromethamine (about 1.40% w/w), water (about 2.5% w/w), lecithin (about 7.5% w/w), simethicone (about 0.02%) and butylated hydroxytoluene (about 0.2% w/w).

In a twenty fifth oil formulation embodiment there is provided a cannabinoid oil formulation for oral administration comprising: CBDA (about 10% w/w), MCT (about % w/w), TPM (about 7.5% w/w), tromethamine (about 1.40% w/w), water (about 2.5% w/w), lecithin (about 7.5% w/w), simethicone (about 0.02%) and butylated hydroxytoluene (about 0.2% w/w).

In a twenty sixth oil formulation embodiment there is provided a cannabinoid oil formulation for oral administration comprising: CBDA (about 1% w/w), MCT (about % w/w), TPM (about 2% w/w), tromethamine (about 1.40% w/w), water (about 2.5% w/w), lecithin (about 7.5% w/w), simethicone (about 0.02%) and butylated hydroxytoluene (about 0.2% w/w).

In a twenty seventh oil formulation embodiment there is provided a cannabinoid oil formulation for oral administration comprising: CBDV (about 10% w/w), MCT (about % w/w), TPM (about 7.5% w/w), tromethamine (about 1.40% w/w), water (about 2.5% w/w), lecithin (about 7.5% w/w), simethicone (about 0.02%) and butylated hydroxytoluene (about 0.2% w/w).

In a twenty eighth oil formulation embodiment there is provided a cannabinoid oil formulation for oral administration comprising: CBDV (about 1% w/w), MCT (about % w/w), TPM (about 2% w/w), tromethamine (about 1.40% w/w), water (about 2.5% w/w), lecithin (about 7.5% w/w), simethicone (about 0.02%) and butylated hydroxytoluene (about 0.2% w/w).

2.2 Capsule Formulations

In a first capsule embodiment there is provided capsule formulations, preferably soft-gel (otherwise known as ‘soft gelatin’) capsules comprising a cannabinoid oil formulation as described under sub-heading 2.1 above.

In a second capsule embodiment, there is provided a capsule for oral consumption comprising:
a cannabinoid oil comprising:
   a cannabinoid;
   a carrier for the cannabinoid in the form of MCT;
   tocopheryl phosphate component in the form of TP and T2P;
   optionally an emulsifier
   optionally a buffering agent
   optionally an aqueous solvent
   optionally an anti-oxidant
   optionally a rheology modifier.
an outer shell comprising gelatin or a starch for encapsulating the cannabinoid oil, the outer shell preferably in the form of a soft-gel.

In the second capsule embodiment, the mass ratio of TP to T2P is about 6:4 to 8:2, preferably about 2:1 respectively, and the mass ratio of the cannabinoid to the tocopheryl phosphate is about 10:1 to 1:10, preferably 5:1 to 1:5, more preferably 2:1 to 1:2. The capsule comprises about 2.5% or less by weight water.

The emulsifier may be utilised to allow homogenous dispersion of water in the oil phase and may be selected from the group consisting of: Neutral surfactants (span and tween) phospholipids (Lecithin, Phospholipon G). The emulsifier may be provided in the minimum amounts required to form a homogenous dispersion of water in oil.

A buffering agent may be utilised to prevent cannabinoid degradation or conversion of one cannabinoid to another cannabinoid, for example to prevent conversion of CBD to THC and may be selected from the group consisting of: tromethamine, Tris, PIPES (piperazine-N,N′-bis(2-ethanesulfonic acid). The buffering agent may be provided in the minimum amounts suitable to maintain the pH of the formulation during the shelf life.

A buffering agent may also be utilised to prevent conversion of one cannabinoid to another cannabinoid and may be selected from the group consisting of: hydrolysed gelatine and pectin. The buffering agent may be provided in an amounts required to prevent ph change during shelf life

An aqueous solvent may be utilised to add water soluble ingredients into the formulation. These may include additional actives or excipients, or buffering agents used to control the pH. The aqueous solvent may be provided minimum amount to dissolve the buffering agent.

An anti-oxidant may be utilised to prevent the oxidation of CBD and may be selected from butylhydroxytoluene, butylatedhydroxyanise, alpha-tocopherol. The anti-oxidant may be provided in an amounts of 0.01-5% w/w.

A rheology modifier may be utilised to prevent the precipitation of the polymeric buffering agents (gelatine, pectin) and maintain formulation homegenity and may be selected from simethicone, alginate, PVP (polyvinyl pyrrolidone). The rheology modifier may be provided in an amounts required to maintain homogeneity.

In the second capsule embodiment, preferably the cannabinoid is a synthetic cannabinoid, more preferably a synthetic cannabidiol (herein CBD) and/or tetrahydrocannabinol (herein THC), the cannabinoid in an amount to provide the capsule with an about 1-250 mg cannabinoid;

In the second capsule embodiment, preferably the MCT is a naturally occurring MCT extract or oil, more preferably a naturally occurring MCT extract or oil that comprises linear or branched alkyl chains comprising 12 or less carbon atoms.

In a third capsule embodiment there is provided a capsule for oral consumption comprising:
a cannabinoid selected from the group consisting of CBG, CBC, CBND, THC, CBN, CBT, CBE, CBL, CBT, CBCN, THCV, CBGA, CBCN, CBDA and CBDV, preferably CBD or THC;
TPM (herein a mixture of mono-(tocopheryl) phosphate (herein TP) and di-(tocopheryl) phosphate (herein T2P))
   wherein the mass ratio of the cannabinoid to TPM is about from 2:1 to 1:2; preferably 1:1;
MCT
   and
wherein the mass ratio of MCT to cannabinoid and TPM is about 1000:3 to
   preferably wherein the capsule comprises about 2.5% or less by weight water.

In a fourth capsule embodiment, there is provided a soft gel capsule comprising an oil formulation comprising: 75 mg CBG, 75 mg of TPM, 700 mg MCT, 75 mg phospholipon 90G, 13.5 mg tromethamine, 21.1 mg purified water, 0.4 mg butylhydroxytoluene, 0.2 mg simethicone, 39.8 hydrolyzed gelatin to provide a total fill weight of 1000 mg.

In a fifth capsule embodiment, there is provided a soft gel capsule comprising an oil formulation comprising: 75 mg CBC, 75 mg of TPM, 700 mg MCT, 75 mg phospholipon 90G, 13.5 mg tromethamine, 21.1 mg purified water, 0.4 mg butylhydroxytoluene, 0.2 mg simethicone, 39.8 hydrolyzed gelatin to provide a total fill weight of 1000 mg.

In a sixth capsule embodiment, there is provided a soft gel capsule comprising an oil formulation comprising: 75 mg CBND, 700 mg MCT, 350 mg glyceryl monolinoleate, mg phospholipon 90G, 13.5 mg tromethamine, 21.1 mg purified water, 0.4 mg butylhydroxytoluene, 0.2 mg simethicone, 39.8 hydrolyzed gelatin to provide a total fill weight of 1000 mg.

In a seventh capsule embodiment, there is provided a soft gel capsule comprising an oil formulation comprising: 75 mg THC, 75 mg of TPM, 700 mg MCT, 75 mg phospholipon 90G, 13.5 mg tromethamine, 21.1 mg purified water, 0.4 mg butylhydroxytoluene, 0.2 mg simethicone, 39.8 hydrolyzed gelatin to provide a total fill weight of 1000 mg.

In an eighth capsule embodiment, there is provided a soft gel capsule comprising an oil formulation comprising: 75 mg CBN, 75 mg of TPM, 700 mg MCT, 75 mg phospholipon 90G, 13.5 mg tromethamine, 21.1 mg purified water, 0.4 mg butylhydroxytoluene, 0.2 mg simethicone, 39.8 hydrolyzed gelatin to provide a total fill weight of 1000 mg.

In a ninth capsule embodiment, there is provided a soft gel capsule comprising an oil formulation comprising: 75 mg CBT, 75 mg of TPM, 700 mg MCT, 75 mg phospholipon 13.5 mg tromethamine, 21.1 mg purified water, 0.4 mg butylhydroxytoluene, 0.2 mg simethicone, 39.8 hydrolyzed gelatin to provide a total fill weight of 1000 mg.

In a tenth capsule embodiment, there is provided a soft gel capsule comprising an oil formulation comprising: 75 mg CBE, 75 mg of TPM, 700 mg MCT, 75 mg phospholipon 90G, 13.5 mg tromethamine, 21.1 mg purified water, 0.4 mg butylhydroxytoluene, 0.2 mg simethicone, 39.8 hydrolyzed gelatin to provide a total fill weight of 1000 mg.

In an eleventh capsule embodiment, there is provided a soft gel capsule comprising an oil formulation comprising: 75 mg CBL, 75 mg of TPM, 700 mg MCT, 75 mg phospholipon 90G, 13.5 mg tromethamine, 21.1 mg purified water, 0.4 mg butylhydroxytoluene, 0.2 mg simethicone, 39.8 hydrolyzed gelatin to provide a total fill weight of 1000 mg.

In a twelfth capsule embodiment, there is provided a soft gel capsule comprising an oil formulation comprising: 75 mg CBCN, 75 mg of TPM, 700 mg MCT, 75 mg phospholipon 90G, 13.5 mg tromethamine, 21.1 mg purified water, 0.4 mg butylhydroxytoluene, 0.2 mg simethicone, 39.8 hydrolyzed gelatin to provide a total fill weight of 1000 mg.

In a thirteenth capsule embodiment, there is provided a soft gel capsule comprising an oil formulation comprising: 75 mg CBD, 75 mg of TPM, 700 mg MCT, 75 mg phospholipon 90G, 13.5 mg tromethamine, 21.1 mg purified water, 0.4 mg butylhydroxytoluene, 0.2 mg simethicone, 39.8 hydrolyzed gelatin to provide a total fill weight of 1000 mg.

In the above described embodiments under this sub-heading, the outer shell may be composed of a gelatin. A gelatin matrix may comprise gelatin, plasticizer, solvent and optional ingredients such as flavors and colorants. Gelatin may arise from a bovine, porcine, or piscine (fish) origin. Gelatin may have a variety of bloom strengths, preferably a bloom strength of 150. Glycerin or sorbitol may be utilised as a plasticizer, preferably glycerin. As an alternative to gelatin, potato starch matrix may be used. Potato starch matrix is a smooth, transparent substance resembling gelatin, which is neutral in taste and color, easily digestible and of plant origin.

2.3 Bioavailability of Oral Administered Cannabinoid

A principal advantage of the invention is that it enables the oral administrative route to achieve higher plasma levels and higher area under curve values of cannabinoid than could be previously obtained at the relevant dose of cannabinoid.

Without wanting to be bound by hypothesis, it is believed that the tocopheryl phosphate component of the oral cannabinoid formulation of the invention, and in particular, the TPM, assists in increasing dispersibility and solubility of cannabinoid in the gastro intestinal tract which leads to increases in bioavailability, or otherwise actuates the minimisation of 1st pass metabolism and excretion of enterally absorbed cannabinoid. This is exemplified in the Examples described below whereby TPM is found in an in vitro model to increase the loading of cannabinoid into an aqueous medium, and to increase the plasma concentration and area under value of cannabinoid in an in vivo animal model.

Surprisingly it has been found that the TPM increases the bioavailability of cannabinoid beyond that obtainable with MCT alone.

Thus, in one embodiment there is provided a method for providing an individual with a plasma concentration of cannabinoid, the method comprising the step of:
oral administration of a formulation of one of Embodiments 1 or 4 to 5 to the individual,
   wherein the plasma concentration of cannabinoid provided in the individual by oral administration of the formulation of Embodiments 1 or 4 to 5 is greater than that obtained by administration of a same or similar dose of cannabinoid in a formulation that does not comprise TPM.

It is also exemplified in the PK profile assessment herein that oral cannabinoid formulations of the invention provide for an extended exposure to pharmacologically effective plasma concentration of cannabinoid. Advantageously, this allows for protection across a longer dosing cycle, which may be from 4 to 8-10 hours.

Thus, in another embodiment there is provided a method of increasing the duration of a cannabinoid in plasma of an individual, the method comprising the step of:
oral administration of a formulation of one of Embodiments 1 or 4 to 5 to the individual,
   wherein the duration of a cannabinoid in plasma of the individual by oral administration of the formulation of Embodiments 1 or 4 to 5 is greater than that obtained by administration of a same or similar dose of cannabinoid in a formulation that does not comprise TPM.

In one embodiment there is provided a method for providing an individual with an increased cMax or AUC of a cannabinoid. The method comprises oral administration of a cannabinoid formulation of Embodiments 1 or 4 to 5 to an individual in need of said increased cMax or AUC. The cMax or AUC that is ordinarily obtained from administration of an oral composition that does not comprise TPM is used as a control to determine the quantum of increase in cMax or AUC of cannabinoid arising from an administration of a formulation of Embodiments 1 or 4 to 5 above. As a result of the administration, the cMax or AUC of cannabinoid in an individual is increased relative to the cMax or AUC of cannabinoid arising from oral administration of a cannabinoid formulation that does not comprise the tocopheryl phosphate component. The quantum of increase is at 4 to 15 times the control.

In one embodiment, cMax or AUC of cannabinoid may be improved by adding TPM to an oil carrier comprising MCT and cannabinoid, or otherwise by increasing the relative amount of TPM in an oil carrier comprising MCT, TPM and cannabinoid. Methods for measuring the plasma concentration and area under curve values of cannabinoid are known to the skilled worker. The examples herein exemplify in vitro methods and an in vivo model for assessing cannabinoid cMax and AUC in formulations containing a range of carrier oil, tocopheryl phosphate and cannabinoid values. These methods can be utilised to assess or otherwise determine improvement in cMax or AUC in cannabinoid-containing compositions arising from relative increases in the amount of one or more of TPM, MCT, cannabinoid in cannabinoid-containing formulations according to the embodiments herein.

2.4 Methods of Treatment

The improved bioavailability of oral administered cannabinoid arising from the formulations of the invention enables the treatment of a range of conditions for which cannabinoids have been suggested. As mentioned herein, some of these conditions include pain, inflammation, anxiety, depression, insomnia, sleep disorders, lack of energy, lack of alertness, weight gain, obesity, diabetes, metabolic syndrome, nausea (acute or anticipatory), epilepsy, spasticity, schizophrenia, bi-polar disorder, cancer and neoplasia, chronic pain, osteoarthritic pain, bacterial and/or fungal infection, fibromyalgia, appetite enhancement and/or appetite suppression.

Thus, in one embodiment there is provided a method of prevention or treatment of one of the above-mentioned conditions comprising the step of administering an oral cannabinoid formulation described herein, thereby preventing or treating an above-mentioned condition.

In another embodiment there is provided an oral cannabinoid formulation for use in the prevention or treatment of one of the above-mentioned conditions.

In another embodiment there is provided a use of an oral cannabinoid formulation described herein for prevention or treatment of one of the above-mentioned conditions.

In another embodiment there is provided a use of an oral cannabinoid formulation described herein, in the manufacture of a medicament for prevention or treatment of one of the above-mentioned conditions.

In a particularly preferred embodiment, the condition is insomnia or other sleep disorder. In this embodiment it is preferred that the formulation is provided in the form of a plurality of dosage units, each individual unit comprising a cannabinoid component that comprises a cannabinoid, preferably CBD or THC in amounts of about 10 to 250 mg/ml and 1-50 mg/ml respectively. The mass ratio of cannabinoid to MCT is about 1:3 to 1:1000 respectively, about 1:3 to 1:500 respectively, or about 1:3 to 1:100 respectively. The mass ratio of TP to T2P is about 2:1, within the range of about 4:1 to about 1:4, or within the range of about 6:4 to about 8:2, and wherein the mass ratio of the cannabinoid to the tocopheryl phosphate component is about 10:1 to 1:10, preferably 5:1 to 1:5, more preferably 2:1 to 1:2. One or more dosage units may be orally administered to the individual from 15 minutes to 1 hour prior to sleep.

In another embodiment, the condition is episodic or chronic and selected from the group consisting of anxiety, depression, epilepsy, spasticity, schizophrenia, bi-polar disorder. In this embodiment it is preferred that the formulation is provided in the form of a plurality of dosage units, each individual unit comprising a cannabinoid component that comprises a cannabinoid, preferably CBD or THC in amounts of about 10 to 250 mg/ml and 1-50 mg/ml respectively. The mass ratio of cannabinoid to MCT is about 1:3 to 1:1000 respectively, about 1:3 to 1:500 respectively, or about 1:3 to 1:100 respectively. The mass ratio of TP of TP to T2P is about 2:1, within the range of about 4:1 to about 1:4, or within the range of about 6:4 to about 8:2, and wherein the mass ratio of the cannabinoid to the tocopheryl phosphate component is about 10:1 to 1:10, preferably 5:1 to 1:5, more preferably 2:1 to 1:2. This may amount to orally administering a dosage unit of the oral cannabinoid formulation to the individual every 4 to 8 hours in which case up to 4 dosage units may be given daily.

In another embodiment, the condition is acute or chronic pain which may be managed by activation of cannabinoid receptors in the individual in need of treatment. Examples include acute pain associated with trauma or surgical intervention, or chronic pain associated with inflammation, osteoarthritis, or neoplasia. In these embodiments, the oral cannabinoid formulation may be given to prevent perception of incident pain, or to manage ongoing pain. In this embodiment it is preferred that the formulation is provided in the form of a plurality of dosage units, each individual unit comprising a cannabinoid component that comprises a cannabinoid, preferably CBD or THC in amounts of about 10 to 250 mg/ml and 1-50 mg/ml respectively. The mass ratio of cannabinoid to MCT is about 1:3 to 1:1000 respectively, about 1:3 to 1:500 respectively, or about 1:3 to 1:100 respectively. The mass ratio of TP to T2P is about 2:1, within the range of about 4:1 to about 1:4, or within the range of about 6:4 to about 8:2, and wherein the mass ratio of the cannabinoid to the tocopheryl phosphate component is about 10:1 to 1:10, preferably to 1:5, more preferably 2:1 to 1:2. The individual may be dosed with the oral cannabinoid formulation of the invention prior to occurrence of pain, or while experiencing pain. The cannabinoid formulation is administered until the individual is no longer in need of pain relief.

The number of dosage units to be given may be determined by individual characteristics including sex, age, weight, other conditions and medications, these factors being with the purview of the skilled worker, and determinable my measuring the plasma level of cannabinoids by standard techniques, including those described above.

2.5 Methods of Manufacture

The invention provides methods for production of the oral cannabinoid formulation of the invention.

A tocopheryl phosphate component comprises TP and T2P.

The combination or mixture of TP and T2P (herein TPM), may be obtained by forming a composition of tocopheryl and P₄O₁₀ and heating the composition to a temperature at which an exothermic reaction occurs between the tocopheryl and P₄O₁₀. This temperature is referred to as an ‘exotherm temperature’. At this point, the temperature of the reaction mixture is allowed to continue to rise and the reaction is completed by the formation of TP and T2P when the temperature of the reaction falls below the exotherm temperature. The phosphorylation of tocopheryl occurs at and above the exotherm temperature. The reaction products may further include poly phosphate complexes. These may be removed by hydrolysis reaction. The process is generally described in WO2018/112512.

Preferably the mass ratio of TP and T2P in the tocopheryl phosphate component is about 10:1 to 1:10, preferably 5:1 to 1:5, more preferably 2:1 to 1:2. A component comprising this ratio may be directly obtained as a product of the above described phosphorylation reaction, by modifying the amount of reaction substrate and or reaction conditions. Alternatively, or additionally, TP or T2P could be added to the product of the phosphorylation reaction to provide the preferred mass ratio of TP to T2P.

The TP and T2P reaction products arising from the above describe phosphorylation reaction are in the acid form and have a pH of about 2 to 4. These reaction products may be added to the formulation as acids, or as a salt (in which case they are neutral), although it is preferred that the reaction products are added as acids.

The mixture of TP and T2P, referred to herein as TPM, arising from the above described reaction process may have a brittle, wax-like or less malleable texture which makes working the TPM with other constituents of the tocopheryl phosphate component (if any) and the cannabinoid component more difficult. To improve the workability of the TPM, an alcohol or other organic solvent may be added to decrease the solid character of TPM. Generally, an alcohol or organic solvent is provided in no more than an amount of about 50% by weight of the tocopheryl phosphate component, or otherwise not more than about 1% by weight of the oral cannabinoid formulation.

As described above, the cannabinoid of the cannabinoid component of the oral cannabinoid formulation may be derived from a synthetic source, or from a natural source, for example a phyto-cannabinoid. It is preferred that it is provided in a form which is miscible with MCT, or dissolvable in oil. In certain embodiments, the cannabinoid may be provided in the form of a powder.

The MCT of the cannabinoid component may be provided in a substantially unextracted form, for example, in the form of a whole oil i.e an oil that contains components derived from the MCT source that are other than MCT. For example, MCT may be provided in the form of a whole palm kernel oil or coconut oil. In certain embodiments it is preferred that MCT is provided as an extract in which the only triglycerides are medium chain—i.e. generally 12 carbons or less. Highly purified extracts of MCT are preferred and may be obtained from a variety of commercial sources.

The MCT generally acts as a carrier for the cannabinoid, which is to say that in one embodiment it bulks the cannabinoid, thereby making working with and formulating the cannabinoid easier. Thus, generally the cannabinoid is provided for use as an ingredient for production of the oral cannabinoid formulation in MCT. The mass ratio of cannabinoid to MCT is about 1:3 to 1:1000 respectively, about 1:3 to 1:500 respectively, or about 1:3 to 1:100 respectively

In embodiments of the manufacture process, the tocopheryl phosphate component is contacted with the cannabinoid component (comprising the MCT carrier/cannabinoid composition) to form the oral cannabinoid composition. This may be achieved by blending the tocopheryl phosphate with the cannabinoid component.

It is important that the blending process should result in the equal and consistent distribution of the TPM throughout the cannabinoid component, ostensibly providing for dissolution of the TPM throughout the cannabinoid component.

In one embodiment, TPM and MCT may be combined and stirred with gentle heating to enable the TPM to dissolve into the MCT to form a first solution of TPM dissolved in MCT. Cannabinoid, which may be in the form of a powder, may then be added to the first solution and mixed to dissolve the cannabinoid into the first solution.

The product of the process may have a range of physical properties, depending on the properties of the tocopheryl phosphate and cannabinoid components utilised as ingredients to form the product, and the reaction conditions. Generally, the product is hydrophobic, or otherwise oil-like in nature.

In one embodiment, the product may be a liquid, such as a liquid oil, and in this form the product may require no further substantial modification, thereby taking the form of the oral cannabinoid formulation that is ready for use. In other embodiments it may be necessary to add reagents to modify viscosity (i.e. to reduce or increase viscosity), depending on whether the oral cannabinoid composition is to take a liquid, solid, or semi solid form. Viscosity modifying agents may be added to either the tocopheryl phosphate or cannabinoid components prior to blending those components to from the oral cannabinoid composition of the invention. Alternatively, these modifying agents may be added after these components have been combined.

The product of the manufacture process may be shaped or molded to form a tablet, caplet, gummie or like chewable confectionery, or suppository.

The product of the manufacture process may encapsulated, for example to form an encapsulated oil, or otherwise coated to form an enteric coating to minimise enteral degradation of the formulation.

A range of other pharmacologically accepted excipients, carriers, flavouring agents, stability modifiers can be added to the product of the manufacture process, or to the tocopheryl phosphate or cannabinoid components before those components are combined.

### EXAMPLES

### Example 1—In Vitro Rat Lipolysis

The concentration in the aqueous phase during intestinal digestion is often presumed to be a parameter for consideration in predicting the likely bioavailability for lipid-based formulations. Intestinal drug precipitation has been proposed as an indicator for poor bioavailability. Change in the nature of solubilising species such as micelles and vesicles in the intestinal aqueous phase is considered an important determinant of ultimate bioavailability for poorly water soluble, but sufficiently permeable drugs administered in lipid based formulations.

In vitro digestion experiments were carried out to determine whether TPM in various carrier oils could increase the aqueous solubility of CBD.

A rat gastric model was selected in order to have best correlation with a subsequent in-vivo pk study in rats. The effect of TPM in an MCT vehicle was assessed by comparison to CBD dissolved in an MCT vehicle at 100 mg/ml.

The in vitro lipolysis experiment simulated rat gastrointestinal conditions. The dispersion study was done in a lipolysis vessel containing simulated rat gastric medium at pH 2.4 and the gastric digestion of the formulations was assessed during 30 min. Afterwards, a concentrated bile buffer together with pancreatin was added to the gastric medium leading to the final concentrations which simulated the rat intestinal conditions. Subsequently, lipid digestion and drug solubilization was evaluated for 60 min.

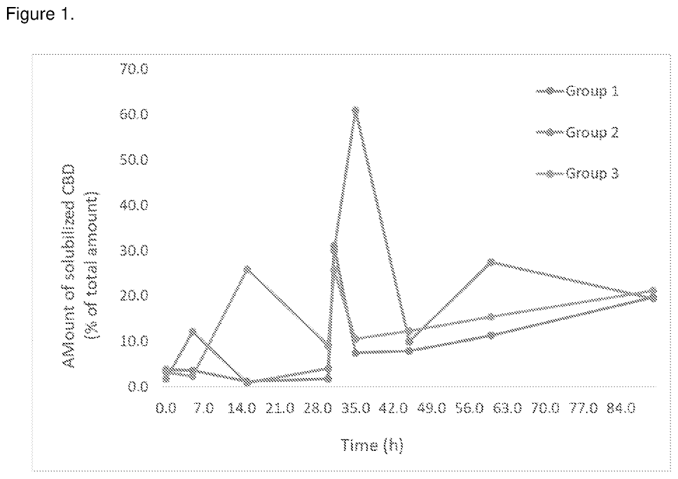

The addition of TPM to MCT containing CBD (100 mg/ml) increased the dispersibility and solubility of CBD in both the simulated gastric and intestinal spaces (FIG. 1). TPM therefore increases the solubility of CBD during digestion in-vitro. This formulation would be predicted to have better bioavailability in-vivo than CBD dissolved in MCT alone.

### Example 3—In Vivo Bioavailability

Formulations that were tested in-vitro for CBD solubility were subsequently tested in-vivo.

Male Sprague-Dawley rats (301-353 g at the time of the study) are acclimatized for a minimum of seven days on standard feed with free access to water. The rats are housed under controlled environmental parameters (temperature: 22.1° C., relative humidity: 57%), and with reversed light cycle (12 h/12 h). Before starting the experiment, the animals are fasted for approximately 13 h in the inactive part of their cycle.

The study included three groups of six rats as shown in the following table:

TABLE 1

| | CBD (mg/ml) | TPM | Mean CBD | |
|---|---|---|---|---|
| Group # | in MCT | (mg/ml) | cMax | AUC |
| Control | 100 | — | 13.9 | 42.5 |
| 1 | 100 | 50 | 78.5 | 178.0 |
| 2 | 100 | 100 | 188.2 | 653.1 |

The formulations are administered to each rat by oral gavage with a polyurethane feeding tube (Instech Laboratories Inc., Plymouth Meeting, USA). Blood samples (200 μL) are collected from the tail vein before dosing and at 0, 0.5, 1, 2, 4, 6, and 8 h after dosing. After the 6 h blood sample, the rats were given access to standard feed. At 23 h, the rats are euthanized by gassing and a blood sample is withdrawn from the heart immediately after. All blood samples were collected in ethylenediaminetetraacetic acid (EDTA) tripotassium salt dihydrate coated tubes (Sarstedt, Helsingborg, Sweden), and centrifuged at 10,000 RPM for 10 min. After centrifugation, the plasma was transferred to polypropylene microtubes and stored at −20° C. until LC-MS analysis.

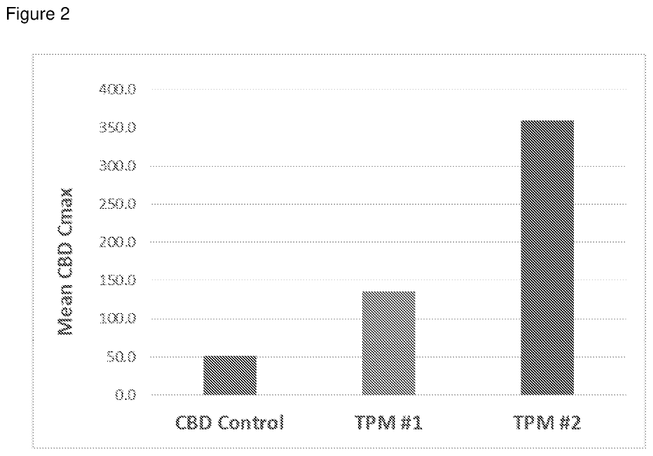

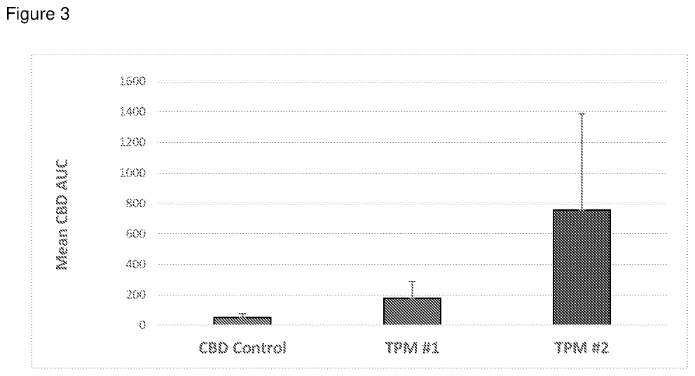

The addition of TPM to MCT carriers increased the mean Cmax relative to the control CBD formulation in MCT alone (FIG. 2). Increases in Cmax were dependent on the TPM concentration. The addition of 50 mg/ml TPM increased the mean Cmax by ˜5×, while the addition of 100 mg/ml TPM increased the mean Cmax by ˜14 times. Similar increases in the area-under-the-curve were evident for the MCT formulations containing TPM (FIG. 3).

The fatty acid chain length plays a key role in the emulsification, permeation, and route of absorption. The medium chain esters are known for rapid, hepatic absorption. The addition of TPM to MCT appears to increase the gastric solubilisation of CBD to enhance its subsequent bioavailability in-vivo.

### Example 4—In Vitro Digestion Model for Assessing Cannabinoid Solubility in Gastric and Intestinal Aqueous Fluid

The solubility of various cannabinoids in formulations according to the invention in gastric and intestinal aqueous fluid is determined by in vitro digestion of cannabinoid formulation in in vitro aqueous gastric and intestinal fluid.

Cannabinoid formulations are prepared by dissolving appropriate amounts of cannabinoid in MCT carrier oil followed by addition of TPM as described in Table 2. Ultrasonication and brief heating in a water bath set to 50° C. is applied to dissolve TPM into the oils.

TABLE 2

| Formulation | Volume | | Cannabinoid | TPM | MCT | Cannabinoid | TPM |
|---|---|---|---|---|---|---|---|
| # | (mL) | Cannabinoid | (mg) | (mg) | (mL) | (mg/mL) | (mg/mL) |
| 1 | 5 | CBG | 125 | 125 | 2.5 | 50 | 50 |
| 2 | 5 | CBC | 125 | 125 | 2.5 | 50 | 50 |
| 3 | 5 | CBND | 125 | 125 | 2.5 | 50 | 50 |
| 4 | 5 | THC | 125 | 125 | 2.5 | 50 | 50 |
| 5 | 5 | CBN | 125 | 125 | 2.5 | 50 | 50 |
| 6 | 5 | CBT | 125 | 125 | 2.5 | 50 | 50 |
| 7 | 5 | CBE | 125 | 125 | 2.5 | 50 | 50 |
| 8 | 5 | CBL | 125 | 125 | 2.5 | 50 | 50 |
| 10 | 5 | CBCN | 125 | 125 | 2.5 | 50 | 50 |
| 11 | 5 | CBD | 125 | 125 | 2.5 | 50 | 50 |
| 12 | 5 | THCV | 125 | 125 | 2.5 | 50 | 50 |
| 13 | 5 | CBGA | 125 | 125 | 2.5 | 50 | 50 |
| 14 | 5 | CBCN | 125 | 125 | 2.5 | 50 | 50 |
| 15 | 5 | CBDA | 125 | 125 | 2.5 | 50 | 50 |
| 16 | 5 | CBDV | 125 | 125 | 2.5 | 50 | 50 |

The gastric medium (pH2.4) is formulated as in Table 3:

TABLE 3

| Composition* | Concentration (mM) |
|---|---|
| NaCl (Merck) | 111.7 |
| Bovine bile (Fluka) | 1.3 |
| Phospholipid (lecithin Lipoid S PC) | 0.8 |

*Added to the composition are 1 mL of pepsin stock (porcine gastric mucosa (450 U/mL of gastric medium) (Sigma-Aldrich) and 1 ml of lipase (Rhizopus Oryzae, (50 TBU/mL of gastric medium) (Sigma-Aldrich)).

A concentrated bile buffer (pH 8.1) is formulated as in Table 4

TABLE 4

| Composition | Concentration (mM) |
|---|---|
| NaCl (Merck) | 133.66 |
| Bovine bile (Fluka) | 64.684 |
| Phospholipid (lecithin Lipoid S PC) | 8.862 |
| Hepes (Sigma) | 47.26 |
| CaCl₂ (Sigma-Aldrich) | 3.892 |

5 mL of cannabinoid formulation is added to 18 mL of gastric medium (pH2.4) and maintained for 5 minutes to enable dispersion. Pepsin and lipase are added to final activities of 450 and 50 U/mL respectively establishing time point zero. Samples are taken at 0, 5, 15 and 30 minutes.

At 30 minutes, concentrated bile buffer and 5 mL pancreatin (Pancreatic lipase (to 179 U/mL of medium) (Sigma-Aldrich) is added to gastric medium to establish an intestinal medium of pH 7.5 as in Table 5.

TABLE 5

| Composition* | Concentration (mM) |
|---|---|
| NaCl (Merck) | 119.6 |
| Bovine bile (Fluka) | 24.1 |
| Phospholipid (lecithin Lipoid S PC) | 3.7 |
| Hepes (Sigma) | 17 |
| CaCl₂ (Sigma-Aldrich) | 1.4 |

Samples of 0.55 mL are taken at 31, 35, 45, 60 and 90 minutes.

An assessment of total sample is done by pipetting 250 μL of homogenous sample into a 1.5 mL Eppendorf tube containing 1000 μL acetonitrile and 225 μL 0.5 M HCl. This is subsequently centrifuged at 10,000 rpm for 5 minutes and the supernatant is analyzed for cannabinoid content using HPLC-UV.

Solubilised drug sample is assessed by adding a homogenous sample of 250 μL wto 7 μL 4-bromobenzene boronic acid solution (1 M in methanol; enzyme inhibitor), and subjected to ultracentrifugation (30 min at 100,000 rpm, 37° C.) in an Optima MAXXP ultracentrifuge (Beckman Coulter, Brea, CA, USA). Subsequently, 200 μL of the supernatant is pipetted into a 1.5 mL Eppendorf tube containing 1000 μL acetonitrile and 225 μL 0.5 M HCl. The Eppendorf tube is centrifuged for 10,000 rpm for 5 minutes and the supernatant analyzed for cannabionid content using HPLC-UV.

Samples are analyzed on a Dionex 3000 HPLC equipment fitted with a Phenomenex Luna, 5μ C18(2), 100A, 150×4.60, 5 μm column (P/NO 00F-4252-E0). Separation is obtained with isocratic elution of an 80:20 mixture of acetonitrile and purified water using the settings in Table 6:

TABLE 6

| Flow: | 0.5 | mL/min |
|---|---|---|
| Column temp: | 30° | |
| Autosampler temp.: | 22° | |
| Detection wavelength | 220 | nm |
| Run time: | 8 | minutes |

The results for each cannabinoid test sample are compared to a matched cannabinoid control which contains the same cannabinoid and carrier oil in same amounts as the test sample but does not contain TPM. An at least 0.5 fold increase in solubility of the cannabinoid as determined by HPLC of the test sample compared to the matched control indicates the sample as having an improved gastro-intestinal solubility and predicts a higher likelihood of improved in vivo gastro-intestinal solubility, higher cMax and or greater AUC than the matched control.

In this model, the amount of cannabinoid to be assessed may be adjusted. Greater amounts of cannabinoid than those stated in Table 2 will generally require a greater amount of TPM (for example greater than 100 mg/mL of TPM or more), or an increased amount of MCT.

### Example 5—Solubility of TPM in Oil Formulations

The solubility of TPM in a range of solvents was determined.

### Experimental

Materials
Labrafac CC [Gattefosse SAS, Saint-Priest France]
Labrafac Lipohile WL 1349 [Gattefosse SAS, Saint-Priest France]
Captex® 300 [Abitec corp, WI, USA]
Captex® 355 [Abitec corp, WI, USA]
PEG 400 [Aldrich, St Louis, MO, USA]
Propylene Glycol [Sigma Aldrich, St Louis, MO, USA]
Glycerol [Merck, Victoria, Australia]
Fractionated oils (MCT Oil from coconut) [Coco earth, NSW, Australia]
Tocopheryl phosphate mixture (TPM) [Avecho Biotechnology, Victoria, Australia]

Equipment
Glass scintillation vials (20 mL) [Rowe Scientific, VIC, Australia]
Teflon-coated Magnetic rods [Rowe Scientific, Victoria, Australia]
Multi-stirrer [Velp, Germany]
Balance (5 digits) (Mettler Toledo Excellence Plus XP205)
Positive Displacement pipettes (Ranin, Mettler Toledo, Vic, Australia)
Positive Displacement pipette tips [Mettler Toledo BioClean Disposable Capillaries/Pistons]
Stainless Steel Spatula [Rowe Scientific, VIC, Australia]
Ratek Water Bath [Rowe Scientific, Victoria, Australia]

Solubility Testing

TPM was accurately weighed into a 20 ml glass scintillation vial according to table 1. A magnetic stirring rod was added to the vial and the total weight recorded (TPM+Vial+magnetic rod). The vials were placed on multi-head magnetic stirrers.

Aliquots of 500 uL solvent were added every 10 minutes to the powder with stirring at 25° C. When the solution was about to become clear the aliquots volume was dropped to 100 uL every 20 minutes. Solvent was added until all TPM powder was completely dissolved. Vials were left overnight at 25° C. without stirring to ensure no crystallisation occur in the solution. The final vial weight was recorded (Total) and used to calculate how much solvent was required to dissolve X gm TPM.

Same procedure was repeated keeping the vials in water bath at 45° C. in order to determine TPM solubility at 40° C.

Results

TPM solubility in the tested solvents is presented in Tables 7 and 8 below. TPM solubility in Captex, Labrafac and MCT oil was between 1.29 to 2.21% w/w. TPM solubility was in PEG 400, Glycerol and Propylene Glycol was less than 0.1% w/w.

Increasing the temperature to 45° C. increased the solubility of TPM in all solvents tested. The maximum solubility increase was observed using Labrafac WL 1349 and the minimum was Glycerol. It was noted that when TPM solutions were brought to room temperature it took less than 30 minutes to show TPM precipitation in PEG 400, Propylene Glycol and Glycerol while it took over 24 hours to show precipitation in the rest of the solvents.

TABLE 7
Solubility of TPM in Various Solvents at 25° C.

| | | TPM | TPM + | | Solvent | Sol |
|---|---|---|---|---|---|---|
| Solvent | Carbons | (gm) | Flea + vial | Total | (gm) | (% w/w) |
| Labrafac | C8 and | 0.212 | 16.444 | 24.389 | 7.944 | 2.67 |
| CC | C10 | | | | | |
| Labrafac | C8 | 0.200 | 15.866 | 23.894 | 8.028 | 2.49 |
| WL 1349 | (70%) | | | | | |
| | C10 | | | | | |
| | (20%) | | | | | |
| Captex ® | C8 | 0.213 | 15.944 | 27.836 | 11.892 | 1.79 |
| 300 | (55%) | | | | | |
| | C10 | | | | | |
| | (35%) | | | | | |
| Captex ® | C8-12 | 0.199 | 15.892 | 24.874 | 8.983 | 2.21 |
| 355 | | | | | | |
| MCT Oil | C8-10 | 0.194 | 15.999 | 30.994 | 14.995 | 1.29 |
| PEG 400 | C3 | 0.020 | 15.691 | 39.043 | 23.352 | 0.08 |
| Propylene | C3 | 0.017 | 16.355 | 39.937 | 23.582 | 0.07 |
| Glycol | | | | | | |
| Glycerol | | 0.016 | 15.659 | 39.542 | 23.883 | 0.07 |

TABLE 8
Solubility of TPM in Various Solvents at 45° C.

| | | TPM + Flea + | | Solvent | Sol |
|---|---|---|---|---|---|
| Solvent | TPM (gm) | vial | Total | (gm) | (% w/w) |
| Labrafac CC | 0.201 | 15.989 | 20.618 | 4.629 | 4.35 |
| Labrafac WL | 0.208 | 15.887 | 20.378 | 4.491 | 4.62 |
| 1349 | | | | | |
| Captex ® | 0.197 | 16.015 | 22.531 | 6.515 | 3.03 |
| 300 | | | | | |
| Captex ® | 0.207 | 16.026 | 22.118 | 6.092 | 3.40 |
| 355 | | | | | |
| MCT Oil | 0.202 | 15.972 | 26.318 | 10.347 | 1.95 |
| PEG 400 | 0.021 | 16.337 | 34.455 | 18.118 | 0.12 |
| Propylene | 0.022 | 15.540 | 32.477 | 16.937 | 0.13 |
| Glycol | | | | | |
| Glycerol | 0.021 | 15.568 | 35.577 | 20.009 | 0.10 |

1. An oral cannabinoid formulation comprising:
a cannabinoid component comprising:
a cannabinoid;
a carrier in the form of medium chain triglyceride (herein MCT);
a tocopheryl phosphate component comprising:
mono-(tocopheryl) phosphate (herein TP) and di-(tocopheryl) phosphate (herein T2P);
wherein the mass ratio of the cannabinoid component to the tocopheryl phosphate component is about 10:1 to 1:10.

2. The formulation of claim 1 wherein the mass ratio of cannabinoid to the tocopheryl phosphate component is about 5:1 to 1:5.

3. The formulation of claim 2 wherein the mass ratio of cannabinoid to the tocopheryl phosphate component is about 2:1 to 1:2.

4. The formulation of any one of the preceding claims wherein the oral cannabinoid formulation comprises about 2.5% or less by weight water.

5. The formulation of any one of the preceding claims wherein the oral cannabinoid formulation comprises cannabinoid in an amount of about 1 to 250 mg/ml.

6. The formulation of any one of the preceding claims wherein the mass ratio of cannabinoid to carrier is about 1:3 to 1:1000 respectively.

7. The formulation of any one of the preceding claims wherein the cannabinoid is cannabidiol (CBD) or tetrahydrocannabinol (THC).

8. The formulation of any one of the preceding claims wherein the mass ratio of TP to T2P is about 2:1.

9. The formulation of any one of the preceding claims wherein the formulation is provided in the form of a plurality of dosage units adapted for oral administration.

10. A dosage unit adapted for oral administration and formed from a formulation of any one of the preceding claims wherein the dosage unit comprise an amount of cannabinoid of about 1 to 250 mg.

11. The dosage unit of claim 10, wherein the unit is a tablet, caplet, capsule, chewable gum or liquid adapted for oral administration.

12. A method for providing an individual with a plasma concentration of cannabinoid, the method comprising the step of:
oral administration of a treatment formulation to an individual, the treatment formulation being a formulation as defined in any one of the preceding claims,
wherein the plasma concentration of cannabinoid provided in the individual by oral administration of the treatment formulation is greater than that obtained by oral administration of a control formulation, wherein the control formulation is the same as the treatment formulation but does not comprise the tocopheryl phosphate component of the treatment formulation.

13. A method of increasing the duration of a therapeutically effective plasma concentration of a cannabinoid in plasma of an individual, the method comprising the step of:
oral administration of a treatment formulation to an individual, the treatment formulation being a formulation as defined in any one of the preceding claims,
wherein the duration of a therapeutically effective plasma concentration of a cannabinoid in plasma in the individual by oral administration of the treatment formulation is greater than that obtained by oral administration of a control formulation, wherein the control formulation is the same as the treatment formulation but does not comprise the tocopheryl phosphate component of the treatment formulation.

14. The method of claim 12 or 13 wherein the cannabinoid is CBD or THC.

15. The method of claim 14 wherein the cannabinoid is CBD.
